(19) United States
(12) Patent Application Publication
(10) Pub. No.: US 2009/0148535 A1
(43) Pub. Date: Jun. 11, 2009

(54) METHOD FOR TREATING CANCER USING INTERFERENCE RNA

(75) Inventor: Cynthia Bamdad, Boston, MA (US)

Correspondence Address: JHK LAW, P.O. BOX 1078, LA CANADA, CA, 91012-1078, US

(73) Assignee: MINERVA BIOTECHNOLOGIES CORPORATION

(21) Appl. No.: 12/315,992

(22) Filed: Dec. 8, 2008

Related U.S. Application Data
(60) Provisional application No. 60/992,943, filed on Dec. 6, 2007.

(51) Int. Cl. A61K 9/51; A61P 35/00

(52) U.S. Cl. 424/499

(57) ABSTRACT

The present application discloses acolloidal nanoparticle that includes a therapeutic nucleic acid species and a targeting protein species attached via a coating on the nanoparticle that facilitates the specific attachment of both species.

### CROSS-REFERENCE To RELATED APPLICATIONS

The present application claims the benefit of priority to U.S. Provisional Patent Application No. 60/992,943, filed Dec. 6, 2007, the contents of which are incorporated by reference herein in their entirety.

### BACKGROUND OF THE INVENTION

1. Field of the Invention

The present invention relates to nanoparticles bearing a cell targeting entity and a therapeutic nucleic acid entity. The present invention also relates to methods of treating cancer as well as proliferating stem cells by contacting the cells with these nanoparticles.

2. General Background and State of the Art

The limited efficacy and nonspecific toxicity of many current cancer therapies has resulted in the demand for better treatments. One approach showing promise for both high efficacy and specificity is the use of interference RNA. Interference RNA can exist as miRNA (micro RNA), some of which are naturally occurring in the body, or they can be small synthetic RNAs called siRNAs (short interfering RNA) and RNAis that can be designed by way of sequence to suppress a targeted genes in vitro or in vivo. Interference RNAs can also be in the form of shRNA (short hairpin RNA) or on a plasmid such that they are replicated in situ. Studies in this area show that interference RNAs can specifically suppress the expression of target genes regardless of disease or condition. Because nucleic acid binding is a function of sequence of the target strand and the complementary strand, it is easy to design nucleic acids that will bind to a specific target nucleic acid or gene. The suppression of genes that are implicated in disease processes are of particular interest. Among that group, genes implicated in the susceptibility or in the progression of cancer are the current focus of many developmental strategies involving RNAi. Because nucleic acids are naturally occurring, therapies based on nucleic acids promise to be effective and relatively non-toxic.

siRNAs are RNA duplexes, typically 19-21 base pairs with 2 unpaired nucleotide overhanging bases, were first discovered in plants, and specifically silence gene expression through specific cleavage of mRNA. These molecules naturally occur in many different cell types, including mammalian cells, and have been implicated in different development and growth processes due to their ability to regulate gene expression. Synthetic siRNAs have been widely used experimentally, and are readily designed with the help of computer algorithms and either commercially synthesized and transfected into cultured cells. In vitro experiments utilizing the highly efficient transfection of siRNA into cultured cells, have yielded large amounts of information on the functions of many different genes, proving to be an invaluable tool in the study of molecular and cellular biology. This has led many to investigate siRNA as a potentially powerful therapeutic agent for a variety of diseases, including cancer.

The major problem that prevents the widespread use of interference RNAs as therapy is that when siRNAs are administered to a live animal, less than 2% of the therapeutic RNA reaches the targeted site. This has led to concern over what the RNAs might be doing at off-target sites and whether or not it is possible to deliver enough of the RNAs to the site where therapeutic intervention is desired. Systemic injection of siRNA into animals does not result in tissue specific gene silencing, primarily because of degradation and nonspecific delivery.

Thus a significant improvement would be the development of specific targeting systems, including those using nanoparticles. The use of magnetic nanoparticles bearing RNAs has shown some success in mice. However, the methods they used would not work in humans. First, magnetic particles are toxic and would not be suitable for use in humans. Secondly, in the mouse experiments, the magnets were physically moved over the mouse's body to concentrate the nanoparticles to a tumor that was xenografted just under the skin surface. Because of unwanted effects of large magnetic fields and the problem of using fields to concentrate the nanoparticles, this approach is not feasible in humans. Other groups have encapsulated siRNAs in various polymer or carbohydrate aggregates, which they call “nanoparticles”, but which are actually just polymer coated siRNA aggregates. Aggregates result from the uncontrolled assembly of molecules and/or polymers. Aggregates are not nanoparticles in the traditional sense of the word but of late many have used the term nanoparticle as a sexy catch phrase. The distinction is important because aggregates have no unifying morphology. They can be formed or separated according to size but each aggregate is physically very different from every other aggregate, in terms of shape, but, more importantly, in terms of surface chemistry. The amount of exposed surface charge or exposed hydrophobicity/hydrophilicity actually depends on the random nature of how that particular aggregate folded. The result is that aggregates or “polymer nanoparticles” are highly irreproducible, so unlikely to become a drug, and also have a high degree of non-specific binding because of the random exposure of charged and/or hydrophobic regions.

Therefore, what is needed are methods to deliver RNAis to the affected site. In order for these methods to be practical for human intervention, they should be reproducible, avoid non-specific binding and have a functionality for delivering siRNAs and another functionality for targeted the delivery vehicle to the affected site.

### SUMMARY OF THE INVENTION

A significant improvement in methods to treat diseases and conditions using interference RNAs or RNAi is to use nanoparticles to deliver the therapeutic nucleic acids to the affected site. Nanoparticles derivatized with functionalized surfaces that bear a nucleic acid to which the RNA can be hybridized and an antibody to target the siRNA to the appropriate cells would be a major improvement over current methods in which experiments show that only about 2% of the RNAi, administered to an animal, reaches the desired site.

Unlike nano-sized aggregates, such as polymers and carbohydrates, true nanoparticles, such as gold colloids, can be made with nanometer control over the diameter of the resultant metal spheres. In addition, methods have been developed to form highly organized and highly reproducible self-assembled monolayer (SAM) coatings on nanoparticles wherein the SAMs can incorporate several functional head groups that could be useful in solving the problem of delivery of therapeutic RNAs. For example, nanoparticle surface coatings that are comprised of molecules terminated in a DNA oligo or an entity that would otherwise specifically bind the therapeutic nucleic acid and a functionality for the specific attachment of a protein constitutes a universal platform for the targeted delivery of RNAi. siRNA can be hybridized to the carrier DNA on the nanoparticle by simple hybridization of a complementary single stranded tail on one of the strands of RNA in the siRNA duplex, or the shRNA, if contained in a plasmid, may be bound to the nanoparticle through a nucleic acid binding entity presented on the nanoparticle.

The nanoparticle can be targeted to specific tissue and cell types by a variety of proteins that can be immobilized on the nanoparticle. For example, antibodies that recognize a cell surface receptor on specific types of cells can be immobilized or covalently attached to the same nanoparticles that bear RNA binding entities, such as a DNA oligonucleotide having specificity to a particular desired RNA sequence for complementary hybridization.

The present invention describes the use of colloidal gold nanoparticles bearing functionalized surfaces that can present an agent that targets the nanoparticle to a specific tissue or cell type and an agent that can carry the therapeutic nucleic acid so that it can be released at the desired site.

In one aspect of the invention, nanoparticles are used as scaffolds for the immobilization of one or more chemical or biological species that are therapeutic agents and wherein the nanoparticle delivers the therapeutic agent to the affected site. In a preferred embodiment the therapeutic agent is a nucleic acid and, still more preferred, it is interference RNA, also called RNAi, siRNA or shRNA. Single stranded miRNA is also contemplated.

In another aspect of the invention, nanoparticles are used as scaffolds for the co-immobilization of one or more chemical or biological species wherein at least one species is a therapeutic agent and at least one species targets the nanoparticle and immobilized therapeutic to the affected site. In a preferred embodiment, the first species is an antibody that targets a co-immobilization interference RNA to a targeted cell or tissue type. The invention also contemplates the use of nanoparticle-immobilized protein ligands to target the nanoparticle to cells expressing the ligand's cognate receptor.

In another aspect of the invention, siRNA or RNAi that specifically suppresses MUC1 is used to modulate the growth of MUC1*-positive cells, such as MUC1*-positive cancer cells or MUC1*-positive stem cells. Similarly, siRNA can be used to suppress proteins that process MUC1, including MUC1 cleavage enzymes MMP14 and TACE, or protein ligands of MUC1*, including NM23.

The present invention also contemplates using RNAi to modulate stem cell growth and differentiation. For example, interference RNAs that suppress MUC1 cleavage enzymes, including MMP-14 and TACE would trigger the onset of differentiation. Similarly, interference RNA that suppresses the MUC1* activating ligand, NM23, would modulate stem cell growth and initiate stages of differentiation.

In another aspect of the invention, RNAi that specifically suppresses MUC1 is delivered to the affected site of unwanted cell growth via co-attachment to nanoparticles that also bear the antibody HERCEPTIN, which acts to target the nanoparticle to HER2-positive cancer cells and in addition, the HERCEPTIN antibody blocks the growth factor receptor function of HER2.

In another aspect of the invention, an antibody fragment such as a single chain antibody or an Fab, or a bispecific antibody is attached to the nanoparticle as the targeting agent and siRNA to suppress a gene is also attached or immobilized on the nanoparticle.

In, yet another aspect of the invention, nanoparticles bearing a targeting agent and carrying a natural or synthetic drug are administered to a patient either systemically or at the affected site.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1A is a cartoon of the experiment and FIG. 1B is photos of NTA-Ni-SAM-coated gold nanoparticles bearing histidine-tagged proteins that have been incubated with micro beads bearing either the cognate antibody or no antibody.

FIG. 2A is a cartoon of the experiment and FIG. 2B is a photo of an agarose gel showing that a DNA oligo was successfully hybridized to nanoparticles bearing SAMs comprised of NTA-Ni⁺⁺—C₁₁, thiols (for the capture of histidine-tagged proteins), tri-ethylene glycol-terminated C₁₁ thiols (to resist non-specific binding), and a DNA-C₁₁ hybrid thiol, to which the oligo was hybridized.

FIGS. 3A-3C show photos of (A) NTA-Ni SAM-coated nanoparticles carrying recombinant, histidine-tagged Glutathione added to a mixture of agarose beads. All but one of the agarose beads in this incubation did not have glutathione on the surface—they were NTA-Ni agarose beads. The red bead (red color is from the agglomeration of nanoparticles onto the bead) is the only bead in this pool that presents the binding partner—glutathione—of the ligand that is on the nanoparticles. (B) A photo of live Human Umbilical Vein Endothelial Cells (HUVECs) that appear red due to binding between a ligand on SAM-coated gold nanoparticles and its cognate receptor on the surface of the cells. (C) As a control, HUVECs were incubated with nanoparticles bearing an irrelevant histidine-tagged peptide of approximately the same size.

FIG. 4 is a photo of a western blot showing that cells to be tested have the gene (GFP) that is to be suppressed and the cell surface receptor (transferrin) that will allow the nanoparticle-attached anti-transferrin to target the co-immobilized siRNA to that specific cell type.

FIGS. 5A-5C show photos of three groups of cells that have been incubated with the nanoparticles of the invention that bear an antibody and siRNA. (A) the cells pictured do not bear the targeted transferrin receptor so the nanoparticles did not bind to these cells; (B) these cells have the tranferrin receptor and have the GFP gene, and the nanoparticles that were added to this group of cells had the siRNA immobilized but no antibody, so no nanoparticles bound to these cells; (C) these cells bore the transferrin receptor and have the GFP gene; nanoparticles added to these target cells bore both anti-transferrin and siRNA specific to suppress GFP; the photo shows nanoparticles bound to most cells (red arrows).

FIG. 6 is a photo of a Western blot that shows that when nanoparticles bearing the antibody to the transferrin receptor and siRNA that suppresses GFP, the expression of GFP protein is suppressed in a concentration dependent manner. Western blots of cells to which either naked nanoparticles or nanoparticles lacking the targeting antibody were added show no suppression of GFP.

FIGS. 7A-7B show photos of two gels after electrophoresis: (A) the nanoparticles used in the experiment pictured in FIG. 5 were loaded onto a protein acrylamide gel and the resultant bands were visualized using a labeled secondary antibody; the bands for the heavy and light chain of the nanoparticle-immobilized antibody confirm that it was properly immobilized on the nanoparticles; (B) the nanoparticles were also analyzed by gel electrophoresis that shows that the the siRNA was also correctly immobilized on the nanoparticles.

FIG. 8 shows a cartoon of how SAM-coated nanoparticles bearing a targeting antibody and immobilized siRNA suppress the gene for green fluorescent protein (GFP) in a specific cell type.

FIGS. 9A-9F show the time course of nanoparticles being added to cells, their agglomeration onto the cells, internalization of the siRNA, subsequent processing of the siRNA within the cell (red fluorescent label cleaved by dicer and other processing enzymes) and subsequent suppression of the GFP, seen as a diminution of the fluorescent green.

FIGS. 10A-10F show Western blots that indicate that breast tumor cells that acquired resistance to the drug HERCEPTIN, upregulated MUC1* but showed no changes in the amount of full-length MUC1 and showed modest reduction of HER2 (target of HERCEPTIN) in one resistant pool but not in the other.

FIG. 11 is a plot of cell growth as a function of HERCEPTIN concentration that shows that the growth of the HERCEPTIN resistant cell pools, BTRes1 and BTRes 2 are no longer inhibited by HERCEPTIN as is the growth of the parent cells, BT474s.

FIGS. 12A-12B show a plot of cell growth as a function of HERCEPTIN concentration that shows that when the MUC1* in the resistant cells is suppressed using a MUC1 specific siRNA, then the therapeutic effect of HERCEPTIN is restored.

FIG. 13 is a plot of cell growth as a function of HERCEPTIN concentration that shows that when the MUC1* in the resistant cells is disabled using an Fab that binds to and blocks MUC1*, then the therapeutic effect of HERCEPTIN is restored.

FIGS. 14A-14C show a series of bar graphs displaying the results of cell growth experiments in which it was demonstrated that acquired resistance to standard chemotherapy drugs was reversed by combination treatment with a MUC1* disabling Fab and the chemo drug.

FIG. 15 is a plot of cell growth of T47D cancer cells that have been transfected with shRNA that suppresses MUC1 expression in response to treatment with HERCEPTIN. The insert is a Western blot that shows how much MUC1* expression was suppressed.

### DETAILED DESCRIPTION OF THE PREFERRED EMBODIMENTS

In the present application, “a” and “an” are used to refer to both single and a plurality of objects.

As used herein, trastuzumab (more commonly known under the trade name HERCEPTIN®) is a humanized monoclonal antibody that acts on the HER2/neu (erbB2) receptor. Trastuzumab's principal use is as an anti-cancer therapy in breast cancer in patients whose tumors over-express (that is, “produce more than the usual amount of”) this receptor. In the present application, Trastuzumab and HERCEPTIN® are used interchangeably. Occasionally herein, HERCEPTIN® may be used without the trademark symbol ®, however, it is understood that HERCEPTIN® is a registered trademark.

The term “MUC1 Growth Factor Receptor” (MGFR) is a functional definition meaning that portion of the MUC1 receptor that interacts with an activating ligand, such as a growth factor or a modifying enzyme such as a cleavage enzyme, to promote cell proliferation. The MGFR region of MUC1 is that extracellular portion that is closest to the cell surface and is defined by most or all of the PSMGFR, as defined below. The MGFR is inclusive of both unmodified peptides and peptides that have undergone enzyme modifications, such as, for example, phosphorylation, glycosylation, etc.

Full-length MUC1 Receptor (Mucin 1 precursor, Genbank Accession number: P15941) is as follows:

(SEQ ID NO:7)
MTPGTQSPFF LLLLLTVLTV VTGSGHASST PGGEKETSAT QRSSVPSSTE
KNAVSMTSSV LSSHSPGSGS STTQGQDVTL APATEPASGS AATWGQDVTS
VPVTRPALGS TTPPAHDVTS APDNKPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDNRPALGS
TAPPVHNVTS ASGSASGSAS TLVHNGTSAR ATTTPASKST PFSIPSHHSD
TPTTLASHST KTDASSTHHS SVPPLTSSNH STSPQLSTGV SFFFLSFHIS
NLQFNSSLED PSTDYYQELQ RDISEMFLQI YKQGGFLGLS NIKFRPGSVV
VQLTLAFREG ITNVHDVETQ FNQYKTEAAS RYNLTISDVS VSDVPFPFSA
QSGAGVPGWG IALLVLVCVL VALAIVYLIA LAVCQCRRKN YGQLDIFPAR
DTYHPMSEYP TYHTHGRYVP PSSTDRSPYE KVSAGNGGSS LSYTNPAVAA ASANL

The term “Primary Sequence of the MUC1 Growth Factor Receptor” (PSMGFR) is a peptide sequence that defines most or all of the MGFR in some cases, and functional variants and fragments of the peptide sequence, as defined below. The PSMGFR may have the following sequence: GTINVHDVETQFNQYKTEAASRYNLTISDVSVSDVPFPFSAQSGA (SEQ ID NO:8) and all functional variants and fragments thereof having any integer value of amino acid substitutions up to 20 (i.e. 1, 2, 3, 4, 5, 6, 7, 8, 9, 10, 11, 12, 13, 14, 15, 16, 17, 18, 19, or 20) and/or any integer value of amino acid additions or deletions up to 20 at its N-terminus and/or C-terminus. A “functional variant or fragment” in the above context refers to such variant or fragment having the ability to specifically bind to, or otherways specifically interact with, ligands that specifically bind to, or otherwise specifically interact with, the peptide of SEQ ID NO:7, while not binding strongly to identical regions of other peptide molecules identical to themselves, such that the peptide molecules would have the ability to aggregate (i.e. self-aggregate) with other identical peptide molecules. One example of a PSMGFR that is a functional variant of the PSMGFR peptide of SEQ NO:8 (referred to as nat-PSMGFR—for “native”) is GTINVHDVETQFNQYKTEAASPYNLTISDVSVSDVPFPFSAQSGA (SEQ NO:9) (referred to as var-PSMGFR, which differs from nat-PSMGFR by including an -SPY-sequence instead of the native -SRY-. Var-PSMGFR may have enhanced conformational stability, when compared to the native form, which may be important for certain applications such as for antibody production. The PSMGFR is inclusive of both unmodified peptides and peptides that have undergone enzyme modifications, such as, for example, phosphorylation, glycosylation, etc.

As used herein, “MUC1*” refers to a shortened form of the MUC1 receptor that includes the cytoplasmic tail and transmembrane portions of MUC1 but having an ectodomain that is terminated after the end of the PSMGFR sequence. This truncated form of the MUC1 receptor stains positive when probed with an antibody raised against the PSMGFR but show no staining when probed with an antibody that binds to the distal portion of the MUC1 receptor, which is most often cleaved and released from the surface of cancer cells.

The term “binding” refers to the interaction between a corresponding pair of molecules that exhibit mutual affinity or binding capacity, typically specific or non-specific binding or interaction, including biochemical, physiological, and/or pharmaceutical interactions. Biological binding defines a type of interaction that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc.

The term “binding partner” refers to a molecule that can undergo binding with a particular molecule. Biological binding partners are examples. For example, Protein A is a binding partner of the biological molecule IgG, and vice versa.

A “ligand” to a cell surface receptor, refers to any substance that can interact with the receptor to temporarily or permanently alter its structure and/or function. Examples include, but are not limited to binding partners of the receptor, (e.g. antibodies or antigen-binding fragments thereof), and agents able to alter the chemical structure of the receptor (e.g. modifying enzymes).

An “activating ligand” refers to a ligand able interact with a receptor to transduce a signal to the cell. Activating ligands can include, but are not limited to, species that effect inductive multimerization of cell surface receptors such as a single molecular species with greater than one active site able to bind to a receptor; a dimer, a tetramer, a higher multimer, a bivalent antibody or bivalent antigen-binding fragment thereof, or a complex comprising a plurality of molecular species. Activating ligands can also include species that modify the receptor such that the receptor then transmits a signal. Enzymes can also be activating ligands when they modify a receptor to make it a new recognition site for other activating ligands, e.g. glycosylases are activating ligands when the addition of carbohydrates enhances the affinity of a ligand for the receptor. Cleavage enzymes are activating ligands when the cleavage product is the more active form of the receptor, e.g. by making a recognition site for a ligand more accessible. In the context of MUC1 tumor cells, an activating ligand can be a species that cleaves MUC1, chemically modifies the receptor, or species that interact with the MGFRs on the surface of the MUC1 tumor cells to transduce a signal to the cell that stimulates proliferation, e.g. a species that effects inductive multimerization.

A “growth factor” refers to a species that may or may not fall into a class of previously-identified growth factors, but which acts as a growth factor in that it acts as an activating ligand.

“Colloids”, as used herein, means nanoparticles, i.e. very small, self-suspendable or fluid-suspendable particles including those made of material that is, e.g., inorganic or organic, polymeric, ceramic, semiconductor, metallic (e.g. gold), non-metallic, crystalline, amorphous, or a combination. Typically, colloid particles used in accordance with the invention are of less than 250 nm cross section in any dimension, more typically less than 100 nm cross section in any dimension, and in most cases are of about 2-30 nm cross section. One class of colloids suitable for use in the invention is 10-30 nm in cross section, and another about 2-10 nm in cross section. As used herein this term includes the definition commonly used in the field of biochemistry.

As used herein, a component that is “immobilized relative to” another component either is fastened to the other component or is indirectly fastened to the other component, e.g., by being fastened to a third component to which the other component also is fastened, or otherwise is transitionally associated with the other component. For example, a signaling entity is immobilized with respect to a binding species if the signaling entity is fastened to the binding species, is fastened to a colloid particle to which the binding species is fastened, is fastened to a dendrimer or polymer to which the binding species is fastened, etc. A colloid particle is immobilized relative to another colloid particle if a species fastened to the surface of the first colloid particle attaches to an entity, and a species on the surface of the second colloid particle attaches to the same entity, where the entity can be a single entity, a complex entity of multiple species, a cell, another particle, etc.

“Signaling entity” means an entity that is capable of indicating its existence in a particular sample or at a particular location. Signaling entities of the invention can be those that are identifiable by the unaided human eye, those that may be invisible in isolation but may be detectable by the unaided human eye if in sufficient quantity (e.g., colloid particles), entities that absorb or emit electromagnetic radiation at a level or within a wavelength range such that they can be readily detected visibly (unaided or with a microscope including an electron microscope or the like), or spectroscopically, entities that can be detected electronically or electrochemically, such as redox-active molecules exhibiting a characteristic oxidation/reduction pattern upon exposure to appropriate activation energy (“electronic signaling entities”), or the like. Examples include dyes, pigments, electroactive molecules such as redox-active molecules, fluorescent moieties (including, by definition, phosphorescent moieties), up-regulating phosphors, chemiluminescent entities, electrochemiluminescent entities, or enzyme-linked signaling moieties including horseradish peroxidase and alkaline phosphatase. “Precursors of signaling entities” are entities that by themselves may not have signaling capability but, upon chemical, electrochemical, electrical, magnetic, or physical interaction with another species, become signaling entities. An example includes a chromophore having the ability to emit radiation within a particular, detectable wavelength only upon chemical interaction with another molecule. Precursors of signaling entities are distinguishable from, but are included within the definition of, “signaling entities” as used herein.

As used herein, “fastened to or adapted to be fastened”, in the context of a species relative to another species or to a surface of an article, means that the species is chemically or biochemically linked via covalent attachment, attachment via specific biological binding (e.g., biotin/streptavidin), coordinative bonding such as chelate/metal binding, or the like. For example, “fastened” in this context includes multiple chemical linkages, multiple chemical/biological linkages, etc., including, but not limited to, a binding species such as a peptide synthesized on a polystyrene bead, a binding species specifically biologically coupled to an antibody which is bound to a protein such as protein A, which is attached to a bead, a binding species that forms a part (via genetic engineering) of a molecule such as GST or Phage, which in turn is specifically biologically bound to a binding partner covalently fastened to a surface (e.g., glutathione in the case of GST), etc. As another example, a moiety covalently linked to a thiol is adapted to be fastened to a gold surface since thiols bind gold covalently. Similarly, a species carrying a metal binding tag is adapted to be fastened to a surface that carries a molecule covalently attached to the surface (such as thiol/gold binding) which molecule also presents a chelate coordinating a metal. A species also is adapted to be fastened to a surface if a surface carries a particular nucleotide sequence, and the species includes a complementary nucleotide sequence.

“Covalently fastened” means fastened via nothing other than one or more covalent bonds. E.g. a species that is covalently coupled, via EDC/NHS chemistry, to a carboxylate-presenting alkyl thiol which is in turn fastened to a gold surface, is covalently fastened to that surface.

“Specifically fastened” or “adapted to be specifically fastened” means a species is chemically or biochemically linked to another specimen or to a surface as described above with respect to the definition of “fastened to or adapted to be fastened”, but excluding all non-specific binding.

Certain embodiments of the invention make use of self-assembled monolayers (SAMs) on surfaces, such as surfaces of colloid particles, and articles such as colloid particles having surfaces coated with SAMs. In one set of preferred embodiments, SAMs formed completely of synthetic molecules completely cover a surface or a region of a surface, e.g. completely cover the surface of a colloid particle. “Synthetic molecule”, in this context, means a molecule that is not naturally occurring, rather, one synthesized under the direction of human or human-created or human-directed control. “Completely cover” in this context, means that there is no portion of the surface or region that directly contacts a protein, antibody, or other species that prevents complete, direct coverage with the SAM. I.e. in preferred embodiments the surface or region includes, across its entirety, a SAM consisting completely of non-naturally-occurring molecules (i.e. synthetic molecules). The SAM can be made up completely of SAM-forming species that form close-packed SAMs at surfaces, or these species in combination with molecular wires or other species able to promote electronic communication through the SAM (including defect-promoting species able to participate in a SAM), or other species able to participate in a SAM, and any combination of these. Preferably, all of the species that participate in the SAM include a functionality that binds, optionally covalently, to the surface, such as a thiol which will bind to a gold surface covalently. A self-assembled monolayer on a surface, in accordance with the invention, can be comprised of a mixture of species (e.g. thiol species when gold is the surface) that can present (expose) essentially any chemical or biological functionality. For example, they can include tri-ethylene glycol-terminated species (e.g. tri-ethylene glycol-terminated thiols) to resist non-specific adsorption, and other species (e.g. thiols) terminating in a binding partner of an affinity tag, e.g. terminating in a chelate that can coordinate a metal such as nitrilotriacetic acid which, when in complex with nickel atoms, captures a metal binding tagged-species such as a histidine-tagged binding species. The present invention provides a method for rigorously controlling the concentration of essentially any chemical or biological species presented on a colloid surface or any other surface. Without this rigorous control over peptide density on each colloid particle, co-immobilized peptides would readily aggregate with each other to form micro-hydrophobic-domains that would catalyze colloid-colloid aggregation in the absence of aggregate-forming species present in a sample. This is an advantage of the present invention, over existing colloid agglutination assays. In many embodiments of the invention the self-assembled monolayer is formed on gold colloid particles.

The kits described herein, contain one or more containers, which can contain compounds such as the species, signaling entities, biomolecules, and/or particles as described. The kits also may contain instructions for mixing, diluting, and/or administrating the compounds. The kits also can include other containers with one or more solvents, surfactants, preservative and/or diluents (e.g. normal saline (0.9% NaCl, or 5% dextrose) as well as containers for mixing, diluting or administering the components to the sample or to the patient in need of such treatment.

The compounds in the kit may be provided as liquid solutions or as dried powders. When the compound provided is a dried powder, the powder may be reconstituted by the addition of a suitable solvent, which also may be provided. Liquid forms of the compounds may be concentrated or ready to use. The solvent will depend on the compound and the mode of use or administration. Suitable solvents are well known for drug compounds and are available in the literature.

The term “cancer”, as used herein, may include but is not limited to: biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Preferred cancers are; breast, prostate, lung, ovarian, colorectal, and brain cancer.

The term “cancer treatment” as described herein, may include but is not limited to: chemotherapy, radiotherapy, adjuvant therapy, or any combination of the aforementioned methods. Aspects of treatment that may vary include, but are not limited to: dosages, timing of administration, or duration or therapy; and may or may not be combined with other treatments, which may also vary in dosage, timing, or duration. Another treatment for cancer is surgery, which can be utilized either alone or in combination with any of the aforementioned treatment methods. One of ordinary skill in the medical arts may determine an appropriate treatment.

A “subject”, as used herein, refers to any mammal (preferably, a human). Examples include a human, non-human primate, cow, horse, pig, sheep, goat, dog, or cat. Generally, the invention is directed toward use with humans.

As used herein, “therapeutic nucleic acid” refers to any nucleic acid that has the effect of regulating the expression of a target gene through hybridization between a portion of the therapeutic nucleic acid and a nucleic acid strand of the target gene. For instance, while therapeutic nucleic acid may act to ameliorate a disease state, the nucleic acid may also act to cause an effect in gene expression that is not related to a disease state.

As used herein, “interference RNA (RNAi)” is any RNA (ribonucleic acid) that has the ability to suppress expression of a target gene by hybridizing to a portion of the mRNA (messenger RNA) that codes for that gene. Such RNA includes, without limitation siRNAs, shRNAs, and micro RNAs.

As used herein, “hybridizing” between the DNA oligo and RNAi is carried out under conditions that are made optimal for such molecules using well-established methods using optimal salt concentration and temperature for hybridization and washing conditions in order to achieve specific binding between these molecules to the extent that therapeutic RNAi reaches its targeted cells or tissue.

As used herein, “targeting entity” or “targeting protein” refers to a protein or a molecule that guides the nanoparticle bearing the targeting entity or targeting protein to the cell or tissue, on which is present the binding counterpart to the targeting protein or targeting entity.

Interference RNA on Nanoparticles

In one embodiment a nanoparticle optionally derivatized with a SAM bears a molecular species and a therapeutic agent such that the molecular species targets the nanoparticle and the immobilized therapeutic to the desired location. The molecular species may include but is not limited to a small molecule, carbohydrate, lectin, protein, peptide, antibody, nucleic acid or nucleic acid derivative. The therapeutic agent may be but is not limited to a drug, a small molecule, protein, toxin, nucleic acid, DNA, RNA, RNAi or micro RNA. The particle need not be particle-like in nature, it may be a polymer or any linker species capable of connecting the molecular species to the therapeutic agent. The particle need not be nano-scale. In a preferred embodiment, the particle is a nanoparticle the composition of which may be, but is not limited to a metal, semi-conductor material, a magnetic material, lipid, liposome or latex.

In a more preferred embodiment the nanoparticle is gold, optionally derivatized with a SAM. In an especially preferred embodiment the molecular species is a protein and the therapeutic agent is nucleic acid. In a still more preferred embodiment, the protein species is an antibody and the nucleic acid is RNAi.

Interference RNA may be immobilized on a nanoparticle either as a linear oligo or duplex of oligos or as a complete plasmid via, for example, binding to a DNA binding domain immobilized first on the nanoparticle. In a preferred embodiment, the antibody that targets the nanoparticle bearing the therapeutic nucleic acid recognizes a receptor that is somewhat specific for cancer cells, e.g. the receptor is overexpressed on cancer cells.

In one embodiment, the antibody binds to a portion of the HER2 receptor and the RNAi suppresses MUC 1. Conversely, the antibody or antibody fragment or antibody derivative binds to MUC1 or the PSMGFR portion of MUC1 and the RNAi suppresses HER2 or other growth factor receptor including but not limited to HER1, HER3, or HER4. Additionally both antibody and RNAi on the same nanoparticle may be targeted to the same receptor, e.g. the targeting antibody may recognize MUC1 and the RNAi may suppress MUC1 expression. The MGFR region of MUC1 acts as a growth factor receptor or co-receptor. Similar to the mechanism of the Her2 receptor, it mediates cell growth via activation of the MAP kinase signaling pathway.

Her2-positive breast cancer cells (T47Ds), which are also MUC1-positive are not killed by treatment with HERCEPTIN, perhaps because both receptors stimulate the same proliferation pathway. It appears that when one entrance to a signaling pathway is blocked, the cell population shifts dependence to another receptor or protein that also accesses the same signaling pathway. Therefore an effective treatment is to simultaneously treat the patient with therapeutics that act on more than one molecule that triggers a particular signaling pathway.

In one embodiment, nanoparticles are used as scaffolds for the immobilization of one or more chemical or biological species that are therapeutic agents and wherein the nanoparticle delivers the therapeutic agent to the affected site wherein the therapeutic agent is a nucleic acid and, still more preferred, it is interference RNA, also called RNAi, siRNA or shRNA. Nanoparticles coated with organized self-assembled monolayers (SAMs) that present nucleic acid binding entities, such as complementary nucleic acids, preferably a DNA oligo, provide an effective method for loading a carrier vehicle with RNAi. DNA is more stable than RNA but RNA will hybridize to the complementary strand of DNA with affinities that are suitable for specific attachment and stable association of the RNAi with the oligo or any other suitable chemical or polypeptide entity that has specific affinity for RNA on the nanoparticle. To load RNAi on a DNA oligo presented on a nanoparticle, one merely needs to design the therapeutic RNA such that it has a region that will hybridize to the DNA oligo on the surface of the nanoparticle. The invention contemplates the use of DNA, PNA, RNA or other nucleic acids and nucleic acid derivatives as carrier agents that are attached to the nanoparticle. A convenient method for presenting DNA oligos on the nanoparticle is to form SAMs from a mixture of thiols some of which are DNA-thiol hybrid molecules. siRNA is then designed with the duplex region sequence specific for the target gene that it is designed to suppress and then a single stranded tail that is designed to hybridize to the exposed DNA incorporated into the SAM.

Nanoparticles bearing DNA, for the attachment of therapeutic RNA, and also bearing an agent that will target the nanoparticle to the affected site would provide a vast improvement over existing methods. One way to target a nanoparticle to a specific cell or tissue type is via the attachment of an antibody or a ligand to the nanoparticle surface. For example, nanoparticles bearing ligands or antibodies to the transferrin receptor which is overexpressed on many cancer cell would target the nanoparticles to those cells. Similarly, nanoparticles bearing antibodies against MUC1, MUC1* an ErbB receptor or any other growth factor receptor would aid in targeting the nanoparticles to cancer cells. Cell surface proteins that also make desirable targets for agents attached to nanoparticles laden with therapeutic agents include PSA, TACE, MMP-14, CEA (carcinoembryonic antigen widely overexpressed in a wide variety of cells), EphA2 (tyrosine kinase receptor whose overexpression is commonly observed in aggressive breast cancers) Urokinase receptor (overexpression is strongly correlated with poor prognosis in a variety of malignant tumors) and CXCR4 (linked to breast cancer invasion and metastasis). Other proteins that make desirable targets for targeted delivery of therapeutic agents include immune system markers such as CD3, CD2, Fc gamma R III activating receptor (CD16) and some superantigens.

The agent attached to the nanoparticle that is a binding partner of these protein targets can be synthetic or natural; a drug, antibody or protein ligand. Protein and peptide ligands of specific receptor targets are contemplated by the invention but antibodies are preferred because of their stability, high affinity and ease of production. Antibodies are attached to nanoparticles of the invention by a number of methods. First, Protein G or Protein A can be attached to the nanoparticles to generate a universal nanoparticle to which any antibody can be instantly attached without coupling steps. One way to do this is to generate a histidine-tagged Protein A or Protein G and immobilize them on nanoparticles via the non-covalent interaction between the histidine tag and an NTA-Ni⁺⁺ moiety incorporated into the nanoparticle surface coating. Another method is to covalently attach the Protein G or Protein A to a nanoparticle via covalent chemistry methods such as EDC/NHS coupling chemistry wherein the surface of the nanoparticle presents exposed carboxylates. In another method, the surface of the nanoparticle presents exposed NHS (N-hydroxy succinamide) moieties or activated NHS-esters that attach any protein species via covalent coupling between the activated NHS and a primary amine (e.g. lysine) on the protein. Similarly, the antibody is often directly attached to the nanoparticle by these methods.

Antibodies can be made with histidine tags and immobilized on NTA-Ni presenting nanoparticles. More preferable is the direct coupling of the antibody to the nanoparticle via covalent coupling between a primary amine on the antibody and an activated NHS-ester on the nanoparticle. EDC/NHS coupling chemistry and similar methods for coupling protein species to a synthetic surface are known to those skilled in the art. Surfaces that present exposed sulfur groups also non-covalently attach proteins via disulfide bonds. Protein or peptide ligands to the targeted receptor are similarly attached to nanoparticles either by non-covalent interaction between a histidine tag on the protein/peptide and NTA-Ni on the nanoparticle or the protein/peptide is covalently attached to the nanoparticle surface.

Methods for attaching therapeutic nucleic acids to the nanoparticle include the incorporation of DNA-thiols and DNA-disulfide molecules into coatings that are formed on gold nanoparticle surfaces. In a preferred method, DNA-thiols or asymmetric DNA-disulfides are mixed with other thiols and SAMs are formed on the nanoparticle surfaces. RNAi that is linear in nature is attached to the resultant nanoparticles by simple hybridization. Plasmid forms of therapeutic nucleic acids and RNAi can be attached to nanoparticles via hybridization or via a protein, like a nucleic acid binding protein or small molecule that binds to the nucleic acid that is to be loaded onto the nanoparticles. In a preferred embodiment, the therapeutic nucleic acid is RNAi in linear form, often called siRNA wherein the sequences that will specifically target that siRNA to the gene to be suppressed are duplex, but wherein one of the RNA strands has an overhanging tail that will hybridize to the DNA tag on the nanoparticle surface.

Nanoparticles bearing targeting antibodies rapidly agglomerate onto cells bearing their cognate receptor. RNAi carried on the nanoparticle is then delivered to the cell having the gene that is to be suppressed. Nanoparticles are in some cases internalized by the target cell wherein the enzymes that process the interference RNA are able to process it while it is still attached to the nanoparticle surface. For instance, it is known that DNA can be amplified by PCR enzymes while it is still attached to the nanoparticle surface.

In other cases, RNAi is released near the target cell and is internalized in that manner. The size of the nanoparticle can be altered to either encourage the internalization of the nanoparticles (small—15-30 nm) or discourage their internalization (larger particles). Similarly, the dissociation of the hybridized therapeutic nucleic acid can be manipulated via base pair mismatch to effect control over how easily the hybridized nucleic acid is released. The invention further contemplates the use of therapeutic RNAs that have unnatural components or are RNA derivatives designed to inhibit the degradation of the RNA. Similarly, unnatural derivatives of the therapeutic RNA that are to be attached to the nanoparticle may be hybridized to a non-naturally occurring nucleic acid derivative molecule that is incorporated into a surface coating on a nanoparticle. Preferred are methods wherein the natural or unnatural carrier nucleic acid is a hybrid molecule wherein one end of the molecule can participate in SAM formation and the other end of the molecule is able to hybridize to the therapeutic RNA.

The suppression of growth factor receptors is an attractive approach for the treatment of cancers. In fact many cancer treatments on the market today are antibodies that bind to and disable cell surface growth factor receptors. A method of the invention is to target nanoparticles to the affected site, e.g. tumor by attaching antibodies to the nanoparticle that are not only targeting devices but they also block the growth promoting action of the growth factor receptor to which they bind. The inhibitory, interfering or micro RNA that the nanoparticles carry may act to suppress the gene that codes for the targeted growth factor receptor or may suppress an entirely different gene or an associated gene.

Cancer cells that acquire resistance to a cancer drug, do so by overexpressing the MUC1* growth factor receptor. Therefore an approach facilitated by the present invention is to administer to a patient, nanoparticles bearing HERCEPTIN, the antibody that disables the HER2 growth factor receptor that drives the growth of breast cancer cells and co-immobilize siRNA to suppress MUC1, the cleaved form of which is the growth factor receptor that also drives the growth of these cells. Alternatively, the nanoparticles can bear antibodies or antibody fragments that recognize MUC1, or MUC1* (the growth factor receptor form), and carry siRNA to suppress the MUC1 gene. Similar strategies are expected to include antibodies or RNAi that act on HER2 co-receptors or MUC1-associated factors such as the cleavage enzymes MMP-14, TACE and/or MUC1 *'s activating ligand, NM23.

The invention also contemplates using nanoparticles bearing antibodies and ligands that target the nanoparticle along with therapeutics that are not nucleic acid-like in nature. In experiments, cancer cells that have developed resistance to standard chemo therapy drugs such as taxol, doxorubicin, cyclophosphamide and the like, overexpress MUC1*. In these cases, antibody or antibody fragments that bind to MUC1* are attached to nanoparticles that also carry the chemo drug and/or RNAi to suppress MUC1.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

Preparation of Self-Assembled Monolayers (SAMs) on gold nanoparticles

Concentrated solutions of gold nanoparticles were prepared by spinning down 40 ml of AuroDye Forte (GE Healthcare, RPN490) in 50 ml ultracentrifuge tubes at 13000 rpm for 30min at 4° C. Next, 37.3 ml of supernatant was carefully removed using electric pipette aid and ˜2.7 ml was left behind. Supernatant was saved. The pellet was resuspended in the remaining ˜2.7 ml using a vortex mixer. The resuspended gold solution was stored at 4° C.

To assemble the SAMs, 400 ul of concentrated gold nanoparticles were placed into each microcentrifuge tube. To this, 400 ul of a solution of a mixture of thiols, optionally having a total thiol concentration from 250 uM to 1 mM, called the “deposition solution”, was added, mixed well and allowed to incubate on the bench top for 2˜3 hrs. The deposition solution consisted of a mixture of thiols some of which were terminated with different functional headgroups, including but not limited to nitrilo tri-acetic acid (NTA), nucleic acids, carboxylates, N-hydroxy succinamide (NHS), activated NHS moieties, NHS-esters, tri-ethylene glycols and other chemically functional headgroups that would facilitate coupling reactions. The relative concentrations of the thiols depends in part on the assay that is to be performed.

To generate NTA-presenting SAMs on gold nanoparticles: Gold nanoparticles (Auro Dye Forte, cat#RPN490V, GE Healthcare, Piscataway, N.J.) was concentrated by spinning 1.5 ml of the particles at 14K for 10′ in microcentrifuge tubes. Supernatant (surfactant) was removed and the pellet was re-suspended in 100 ul of the supernatant. Next, thiol deposition mixture was prepared in DMF as follows: 30 uM NTA (Nitrilo tri acetic acid)-thiol, and 570 uM COOH-thiol. Total thiol concentrations totaled 600 uM. Next, 100 uL of the deposition solution was added to each tube of the re-suspended colloid pellet, mixed, and allowed to incubate at room temperature for at least 2 hours. Nanoparticles were pelleted at 14K rpm for 10′ in a microcentrifuge. Supernatant was removed and the pellet re-suspended in the 200 uL surfactant.

A 400 uM solution of tri-ethylene glycol (EG3)-terminated thiol was made in DMF and 400 uL of this solution was added to each tube of re-suspended pellet. This was followed by heat cycling as follows: 2 min at 55° C., 2 min at 37° C., 1 min at 55° C. and 2 min a tubes were allowed to cool to room temperature for 5 minutes.

The nanoparticles were then spun at 14K rpm for 10′ in a microcentrifuge. To the pellet was added 600 uL of Ni-Surfactant solution (95 uL 1% NiSO4 into 20 cc surfactant), the tube was mixed and allowed to incubate for 5′ at room temperature. Next the nanoparticles were pelleted by spinning at 14K rpm for 10′. Supernatant was removed and pellet re-suspended in 1 ml of phosphate buffered saline. The nanoparticles were stored at 4° C. To attach a histidine-tagged protein or peptide, an aliquot of the desired peptide was added to NTA-Ni⁺⁺ nanoparticles in phosphate buffer or PBS. If desired, centrifugation is used to remove excess peptide.

To generate DNA-presenting SAMs on nanoparticles: Gold nanoparticles (Auro Dye Forte, cat#RPN490V, GE Healthcare, Piscataway, N.J.) were concentrated by spinning 1.5 ml of the particles at 14K for 10′ in microcentrifuge tubes. Supernatant (surfactant) was removed and the pellet was re-suspended in 100 ul of the supernatant. Next, thiol deposition mixture was prepared in DMF as follows: 30 uM NTA (Nitrilo tri acetic acid)-thiol, 10 uM DNA-thiol and 560 uM COOH-thiol. Total thiol concentrations totaled 600 uM. Next, 100 uL of the deposition solution was added to each tube of the re-suspended colloid pellet, mixed, and allowed to incubate at room temperature for at least 2 hours. Nanoparticles were pelleted at 14K rpm for 10′ in a microcentrifuge. Supernatant was removed and the pellet re-suspended the in 200 uL surfactant.

A 400 uM solution of ethyleneglycol-terminated thiol (EG3) was made in DMF and 400 uL of this solution was added to each tube of re-suspended pellet. This was followed by heat cycling as follows: 2 min at 55° C., 2 min at 37° C., 1 min at 55° C. and 2 min at 37° C. Next, the tubes were allowed to cool to room temperature for 5 minutes.

The nanoparticles were then spun at 14K rpm for 10′ in a microcentrifuge. To the pellet was added 600 uL of Ni-Surfactant solution (95 uL 1% NiSO4 into 20 cc surfactant), the tube was mixed and allowed to incubate for 5′ at room temperature. Next the nanoparticles were pelleted by spinning at 14K rpm for 10′. Supernatant was removed and pellet re-suspended in 1 ml of phosphate buffered saline. The nanoparticles were stored at stored at 4° C.

To the DNA thiol bearing nanoparticles (DNA-Au-NP), another DNA molecule (165 nucleotide long) was attached through hybridization as follows. To 50 ul DNA-Au-NP, 100 nM-1 uM short oligo was added and annealed at 94 C/1 min, 52 C/1 min, 37 C/1 min, and RT/5-10 min. This was followed by a spin and wash with 1 ml PBS. The final pellet was re-suspended in 50 ul of phosphate buffered saline. (See FIG. 2B). Essentially any nucleic acid can be hybridized to the oligo tag by methods known to those skilled in the art.

To generate SAMs on gold nanoparticles comprised of DNA-thiols NTA-thiols NHS-ester-thiols and tri-ethylene glycol thiols: One deposition solution that was used was 150 uM COOH-thiol, 50 uM NTA-thiol, 100 uM NHS-ester-thiol, and 300 uM DNA-thiol. SAMs that were comprised of NHS-ester-thiols and DNA-thiols were prepared from a DMF solution containing 100 uM NHS-ester-thiol, and 500 uM DNA-thiol. The chemical formula of one DNA-thiol used was:

C₁₁—S—S—C₁₁-EG₃-NH-5′-GTC AGT CAG TCA GTC-3′ (SEQ ID NO:1) (wherein EG₃ stands for 3 ethylene glycol units).

Following incubation of the naked gold nanoparticles in deposition solution, tri-ethylene glycol-terminated-(C₁₇H₃₆O₄S)-thiol solution (400 μM ) was added into the mixture and was heat cycled or placed in a water bath as follows: 55° C. for 5 min, 37° C. for 5 min, 55° C. for 5 min, and 37° C. for 5 min. SAM coated gold nanoparticles were allowed to cool down to RT (5˜10). The nanoparticle solution was then centrifuged to a soft pellet, then resuspended in a phosphate buffer until further use. Nanoparticles bearing NTA, were incubated in 0.25% nickel sulfate solution in water, then centrifuged to a soft pellet, and resuspended and stored in normal phosphate buffer.

To prepare SAMs on nanoparticles that presented both an oligo nucleotide and a functional group for the attachment of a targeting antibody: nanoparticles were coated with SAMs that presented an activated NHS group and a DNA oligo as described above. In some cases, NTA-Ni was also incorporated.

Method 1 for attaching an antibody to SAM-coated nanoparticles bearing an activated NHS moiety: The DNA-NHS-SAM coated nanoparticles (described directly above) were spun down at max speed for 10 min in a microcentrifuge and the supernatant discarded. A solution containing anti-transferrin receptor antibody(3B8 2A1, Santa Cruz, Santa Cruz) and Cy3 GFP specific siRNA (Dharmacon, Lafayette, Colo.) in 1 ml of Na-phosphate buffer (100 mM, pH 7.4) was added to the nanoparticle pellet. The antibody should be added immediately after the spinning step because NHS loses activity quickly in aqueous solution.

To attach siRNA to the nanoparticles: Up to 50 nM of antibody and 10 μM of tagged siRNA can be loaded onto 1 ml of NP. Next, the mixture was incubated at 37° C. for 2 hrs, followed by the addition of ethanolamine to a final concentration 1 mM into each tube to quench the reaction. The coupling was allowed to take place either at RT for 2 hrs or at 4° C. overnight. Nanoparticles were then spun at maximum speed for 10 min. Supernatant was discarded and 1 ml of fresh phosphate buffered saline was added and the nanoparticles resuspended with repeated pipetting and stored at 4° C.

The sequence of the GFP specific siRNA that was hybridized to the DNA carrier tag was:

EGFP sense (Cy3 labeled - fluorescent red color)

(SEQ ID NO:2)

5′-GCA AGC TGA CCC TGA AGT TGT T-GTC AGT CAG TCA
GTC-3′
EGFP antisense

(SEQ ID NO:3)

5′-GAA CTT CAG GGT CAG CTT GCT T-3′

The underlined portion of the EGFP sense RNA is the region that hybridizes with the DNA in the DNA thiol.

Method 2 for attaching an antibody to SAM-coated nanoparticles bearing DNA and NTA-Ni⁺⁺: Antibodies were also attached to nanoparticles via a recombinant histidine-tagged single domain protein G (proG-His). The His-tagged Protein G was first immobilized on the nanoparticles via the non-covalent interaction between NTA-Ni⁺⁺ and the histidine-tag on the Protein G. To attach any antibody, one merely needs to add an aliquot of the antibody to the nanoparticle solution in PBS. The resultant nanoparticles may be centrifuged to remove excess antibody or the desired amount may be added if centrifugation is to be avoided. The Protein G was expressed in E. coli and purified by NTA-agarose chromatography. S. aeruginosa genomic DNA was used as template for PCR amplification for the generation of the single domain protein G. The cloning primers were

5′ primer 5′-AAA GCA GGC TCC ACC ATG AAA CCA GAA GTG ATC GAT GCG (SEQ ID NO:4) and 3′ primer was 5′-ACA AGA AAG CTG GGT CCT ATG TCA CGG CTG GTG TTA ATT CAG (SEQ ID NO:5). Gateway expression system (Invitrogen, Carlsbad, Calif.) was used to make the expression construct using the expression vector pEXP2-DEST.

### Example 2

Experiment demonstrating the specificity of nanoparticles coated with affinity functionalized SAMs.

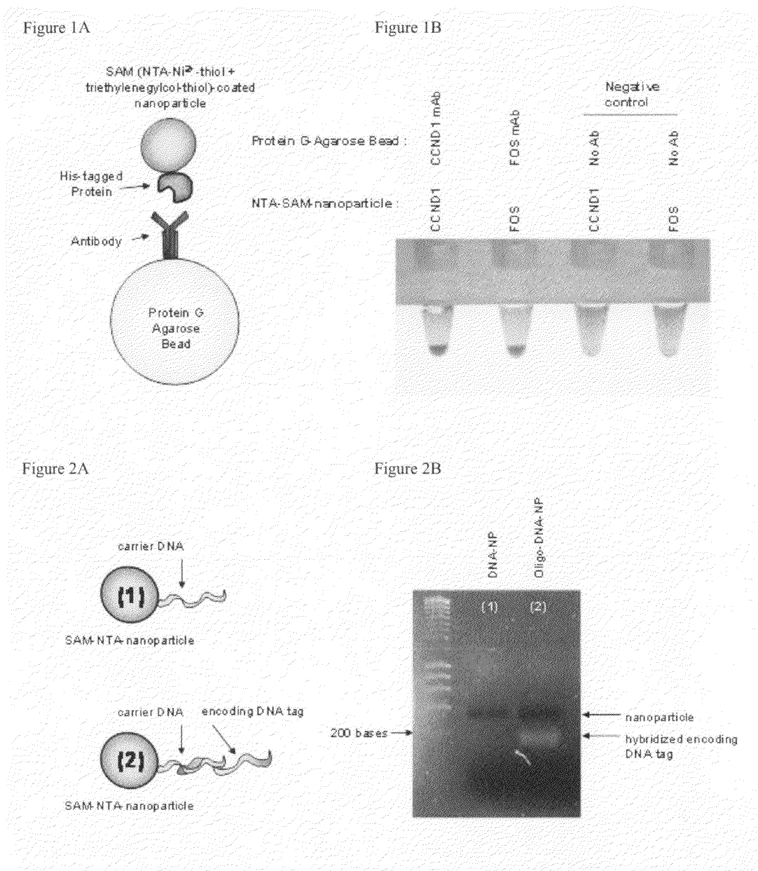

SAM-coated nanoparticles, with proteins immobilized via interaction between a histidine tag on the protein and NTA-Ni incorporated into the SAM, specifically bind their target and show virtually no non-specific binding. Cognate antibodies were bound to micro-scale, protein G derivatized agarose beads. NTA-Ni-SAM-nanoparticles with immobilized CCND1 protein or Fos protein were mixed with the antibody-bearing beads. Beads and nanoparticles were combined in PBS and incubated at 4 C on a rotary shaker. Samples were removed, agarose beads were allowed to settle due to gravity, and were photographed, (see FIG. 1). Micron-scale beads are pulled out of solution by gravity, but free 15-50 mn colloidal particles in a homogeneous suspension do not settle due to gravity. In the control experiments wherein no antibody was attached to the beads, the beads settled but remained clear in color because no protein-bearing nanoparticles had bound to them.

### Example 3

Preparation and performance of SAMs incorporating both NTA-Ni-thiols, tri ethylene glycol-terminated thiols and DNA-thiols. SAMs were formed on nanoparticles as described above. A 165 base DNA tag was hybridized to carrier DNA-thiols incorporated into the SAMs. The nanoparticles were washed three times with PBS. Nanoparticles before (Lane 1) and after (Lane 2) hybridization with the DNA tag were resolved on a 1% agarose gel for detection of the DNA tag. The DNA band at the appropriate size is clearly visible in the lane that had the hybridized DNA and absent in the lane where DNA was not hybridized to the oligo tag in the SAM, (See FIG. 2).

### Example 4

Proteins immobilized on SAM-coated nanoparticles resist non-specific binding and bind only to beads or cells that present the cognate receptor for the ligand attached to the nanoparticle.

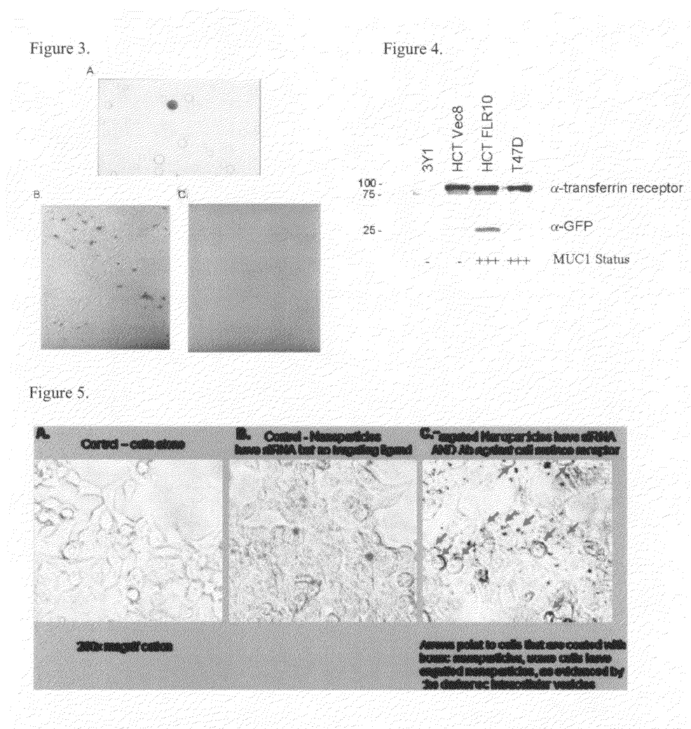

NTA-Ni SAM-coated nanoparticles were incubated with recombinant, histidine-tagged Glutathione-S-Transferase (GST) protein. Nanoparticles were washed to remove unbound GST. A single agarose bead derivatized with Glutathione, which is the binding partner of GST, was dropped into a pool of agarose beads that did not bear Glutathione. The nanoparticles bearing GST protein were incubated with the mixed pool of beads at 4° C. overnight. The result was that the GST-bearing nanoparticles bound to the single bead that bore its binding partner Glutathione. The red bead (red color is from the agglomeration of nanoparticles onto the bead) is the only bead in this pool that presents the binding partner—glutathione—of the ligand that is on the nanoparticles. This demonstrates high specificity and essentially no release of the nanoparticle-immobilized protein (See FIG. 3A).

Similarly, a histidine-tagged peptide derived from vitronectin was attached to NTA-Ni-SAM coated nanoparticles. As a control, an irrelevant histidine-tagged peptide of comparable size was attached to another pool of the nanoparticles. Both nanoparticle types were then incubated with live Human Umbilical Vein Endothelial Cells (HUVECs) that are rich in alphav-beta3 receptors. Vitronectin binds to the alphav-beta3 receptor. The nanoparticle-cell mixtures were incubated for several hours in a CO₂ incubator. The result were examined and photographed under a microscope without any wash steps. The result was that the nanoparticles bearing the ligand for a receptor on the surface of the cells, agglomerated onto the cells and colored them red (from the gold). The same cells that were incubated with nanoparticles bearing an irrelevant peptide did not (See FIGS. 3B, C).

### Example 5

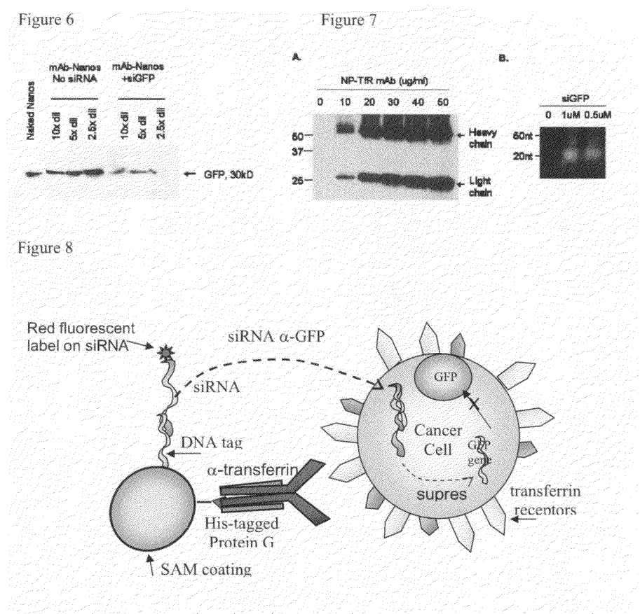

Confirmation of specific antibody and siRNA presence on the nanoparticles. (A) An increasing amount of antibody for transferrin receptor (TfR) was added to the NHS-ester-coated nanoparticles (pre-siRNA loaded) and antibody presence was detected using a HRP-labeled secondary antibody. (B) An increasing amount of siRNA against GFP was added to the carrier DNA-thiol-coated nanoparticles (pre-mAb loaded) and its presence was detected with 1× GelStar solution after a 10% polyacrylamide gel electrophoresis. (See FIG. 7).

### Example 6

Targeted suppression of GFP by nanoparticle delivery of GFP specific siRNA that was targeted to the desired cells via an antibody that was co-immobilized on the nanoparticle along with the siRNA.

FIG. 8 is a cartoon depicting the strategy for the experiment that follows. In short, nanoparticles bearing a transferrin receptor and also carrying siRNA specific for GFP, which has been transfected into cells bearing transferrin receptors, deliver the siRNA into the targeted cells where time course photos show that the siRNA is processed inside the cells (a red fluorescent label at one end is cleaved as the dicer enzymes process) and the green fluorescence from GFP goes away.

Preparation of Cells that had GFP (Green Fluorescent Protein) Inside and Transferrin Receptors on the Outside

Full-length MUC1 cDNA was assembled by piecing together DNA from different EST clones obtained from ATCC (American Type Culture Collection, Manassas, Va.) and from RTPCR carried out on total RNA from T47D cells. The final coding sequence containing 41 repeats was cloned in between the EcoRI and BamHI sites of the plasmid pIRES2-EGFP (Clontech, Mountain View, Calif.). A MUC1* expression construct was made by cloning in frame the sequence corresponding to the C-terminal 145 amino acids of MUC1 beginning at GTNV . . . and ending at . . . AAAASANL after the nucleotide sequence for the signal peptide. In this way, one vector plasmid coded for MUC1 * and GFP (green fluorescent protein) which were then used in siRNA nanoparticle experiments. The vector plasmids were transfected into HCT116 cells and stable transfectants were selected by using G418 at concentration of 600 μg/ml in DMEM medium. Cells were sorted and maintained as single cell clones. HCT116-MUC1* clone #10 cells (HCT116-MUC1*-10) were used in the nanoparticle targeting experiments detailed below.

Characterization of the Engineered Cells

Lysates from normal (non-cancerous) rat 3Y1 fibroblasts, HCT116 cells transfected with pIRES2-eGFP with (HCTFLR10) or without (HCTVec8) the MUC1 transgene, and the human breast tumor line T47D were analyzed by Western Blot for the presence of the transferrin receptor and GFP expression. FIG. 4 is a photo of a western blot showing that cells to be tested have the gene (GFP) that is to be suppressed and the cell surface receptor (transferrin) that will allow the nanoparticle-attached anti-transferrin to target the co-immobilized siRNA to that specific cell type.

siRNA specific for GFP was hybridized, as described above, to DNA-SAM-coated nanoparticles to which anti-transferrin antibody had also been covalently attached via NHS mediated covalent coupling (described above as Method 1 for antibody attachment to nanoparticles).

Targeted Nanoparticle Delivery of siRNA Specific for GFP

FIG. 5 shows that nanoparticles bearing the targeting antibody and the siRNA do not localize on control cells that do not have the transferrin receptor (A). Nanoparticles bearing the siRNA but not the targeting antibody did not bind to or localize on the target cells that did have the transferrin receptor (B). However, nanoparticles bearing both the targeting anti-transferrin and the siRNA localized to the targeted cells and in some cases, nanoparticles were internalized. FIG. 6 shows that nanoparticles bearing the targeting antibody (“mab” in figure) but no siRNA did not suppress the expression of GFP when added to the target cells and the control cells. However, nanoparticles bearing the targeting antibody and siRNA for GFP suppressed GFP expression in a concentration dependent manner.

Time Course Photos of Targeted Suppression of GFP

5×10⁵ HCT116-MUC1*-10 cells were plated in several 35 mm cell culture dishes on collagen-coated cover glass (Biocoat™; BD Biosciences, San Jose, Calif.). Forty-eight hours later, medium was removed, and cells were incubated at 4° C. in 1 ml PBS for 10 minutes. PBS was removed. Nanoparticles bearing the antibody to the transferrin receptor and bearing siRNA specific for GFP were added to the cell cultures. The nanoparticles were prepared as described above. The nanoparticles diluted 1:5 in PBS (1 ml total volume) were added to cells. Nanoparticles bearing both anti-transferrin receptor and siRNA for GFP were added to 6 dishes for incubation times of 0′, 10′, 30′, 1 hour, 2 hours, and 24 hours, and negative control nanoparticles (without antibody) were added to 3 dishes for 0′, 30′, or 2 hours. No nanoparticles were added to an additional dish, for another control. Plates were incubated at 4° C. for 30 minutes; during this time, plates were tilted every 10 minutes to redistribute nanoparticles. Solutions were removed from each 35 mm dish by aspiration, and plates were washed twice for five minutes at 4° C. in cold PBS. PBS was removed from the dishes. One ml of 4% paraformaldehyde (Sigma-Aldrich, Saint Louis, Mo., pre-dissolved in PBS was added to the 0′ incubation dishes, plus the control (no nanoparticle) dish. Cells were fixed for five minutes at 4° C., and washed once in PBS. Dishes were kept in PBS at 4° C. until the end of the internalization experiment. To the remainder of the dishes, 2 ml of medium prewanned to 37° C. was added. After 10′, 30′, 1 hour, 2 hours, and 24 hours internalization with Transferrin Receptor-loaded nanoparticles, or 30′ and 2 hours internalization with control nanoparticles, dishes were washed once in cold PBS for 4° C. briefly, and fixed for five minutes in 4% paraformaldehyde at 4° C., washed once in cold PBS, and kept in wash at 4° C. until the end last time point.

Cells on cover glass were then blocked and permeabilized with 0.1% NP-40, 2.5% BSA, 2.5% FBS diluted in PBS for 30 minutes at 4° C. Cells were then hybridized with Alexa 633-conjugated Anti-Mouse antibody (Invitrogen, Carlsbad, Calif.) diluted 1:200 in 0.1% NP-40, 2.5% BSA, 2.5% FBS in PBS for 30′ at 4° C. Cells were washed 3×5′ in PBS, then mounted on glass microscope slides using Anti-Fade Mounting Media (Biomeda, Foster City, Calif.). Cover glass was sealed using clear nail polish, and cells were visualized, and images were taken using a Nikon TE2000-U inverted Microscope with the C1 confocal system (Nikon, Tokyo, Japan).

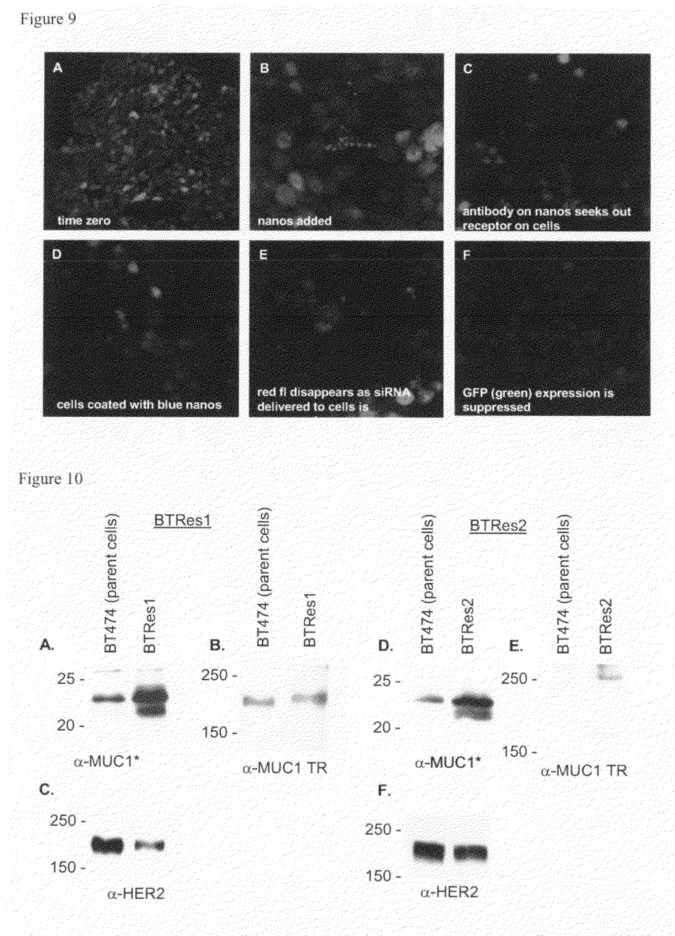

FIG. 9 is comprised of 6 panels showing the time course of nanoparticles being added to cells, their agglomeration onto the cells, internalization of the siRNA, subsequent processing of the siRNA within the cell (red fluorescent label cleaved by dicer and other processing enzymes) and subsequent suppression of the GFP, seen as a diminution of the fluorescent green. It can be seen in the photos that when the nanoparticles are first added to the cells, there are clumps of fluorescent blue and red indicating that the nanoparticles are not yet targeting specific cells. Note that a fluorescent blue secondary antibody is added for visualization just prior to imaging; a red fluorescent tag is attached to the end of one of the siRNA strands and its is clipped off inside the cell by the RNA processing enzymes. After some time, fluorescent blue halos (and to lesser degree red) around the cells that bear the target receptor are present, indicating that the nanoparticles bound to the cells bearing the target receptor. at a later time point, red fluorescence can be seen inside the cells, then it disappears, indicating that the siRNA was internalized by the cells and that it was processed by the dicer and associated enzymes. Finally, at a later time point, no green fluorescence from the GFP can be detected, indicating that the delivered siRNA suppressed the target gene, GFP.

### Example 7

Inducing HERCEPTIN resistance in vitro and subsequent characterization.

Two pools of BT474 cells (BTRes1 and BTRes2) were made resistant by culturing in the presence of 1 ug/ml HERCEPTIN (Trastuzumab; Genentech) for 3 months. Western blots of BT474 breast cancer cells and cells that were induced to become HERCEPTIN resistant, BTRes1 and BTRes2, show a dramatic increase (4-6×) in the expression of MUC1* (A, D) but not the full-length MUC1 protein (B, E). Levels of the HER2 receptor, which HERCEPTIN targets, were only reduced by 18-25% (C, F). (See FIG. 10)

### Example 8

HERCEPTIN resistant cells overexpress MUC1* and no longer inhibited by HERCEPTIN.

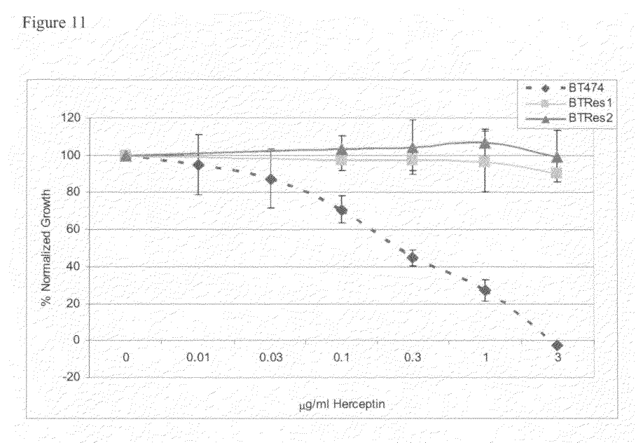

BT474 breast cancer cells become immune to the therapeutic effects of HERCEPTIN following long-term growth in the presence of 1 ug/ml of the antibody. Treatment of HER2-positive BT474 cells with increasing amounts of HERCEPTIN results in a dose-dependent inhibition of cell growth, as determined by cell counts three days post-treatment (dashed line). The growth of two separate populations of BT474 cells with induced HERCEPTIN resistance, BTRes1 and BTRes2 (upper two solid lines), is unaffected by treatment with HERCEPTIN, (see FIG. 11). The experiments were performed as follows.

Growth of BT474, BTRes1 and BTRes2 cells in HERCEPTIN

Cells were plated in 96 well plates at 10,000 cells/well, six wells/condition. The following day, zero hour counts were taken, and medium was changed in the remaining wells to RPMI containing HERCEPTIN to final concentrations of 0, 0.01, 0.03, 0.1, 0.3, 1, and 3 ug/ml. Three days later, the remaining cells were counted using a hemocytometer.

Percent Normalized Growth was then calculated: Percent Normalized Growth=[(Day 3 cell counts with Antibody Added )−(Zero-day cell counts)]/[(Day 3 Cell counts without antibody added)−(Zero-day cell counts)]×100%

### Example 9

siRNA specific for MUC1 restores the therapeutic effect of HERCEPTIN

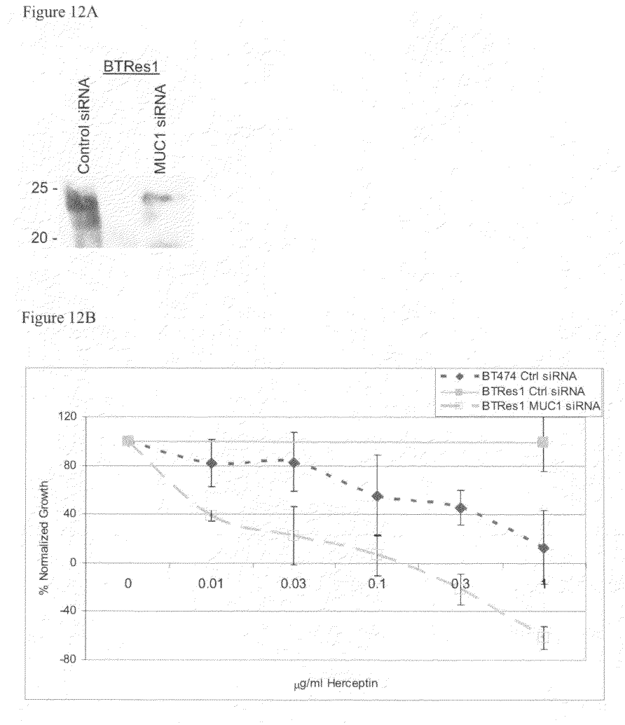

Down regulation of MUC1 by siRNA sensitizes HERCEPTIN resistant BT474 cells to growth inhibition mediated by HERCEPTIN. BTRes1 cells were transiently transfected with a MUC1-specific or a control siRNA, and growth of these cells in the presence of HERCEPTIN was compared with growth of BT474 cells transfected with control siRNA by cell counts three days post-treatment with HERCEPTIN. Growth of BTRes1 cells transfected with control siRNA (solid line) is unaffected by HERCEPTIN treatment, but growth of BTRes1 cells transfected with MUC1 siRNA is reduced (dashed line). Growth of BT474 cells transiently transfected with a control siRNA (dotted line) is essentially unchanged from previous experiments where siRNA was not present. Down regulation of MUC1* was demonstrated by Western Blot (right) (See FIG. 12). The experiments were performed as follows.

Growth of BT474 and BTRes1 Cells Transfected with Either MUC1-Specific siRNA or a Control siRNA in the Presence of HERCEPTIN

BT474 cells were transfected in triplicate with control siRNA (Santa Cruz Biotechnologies; sc-37007), and BTRes1 cells were transfected in triplicate with control or MUC1-specific siRNA (Santa Cruz Biotechnologies sc-35985). One microliter of 10 uM siRNA was added to 100 ul of OptiMEM medium (Invitrogen 22600134). Six microliters of HiPerfect reagent (Qiagen 301704) were then added to each tube. After vortexing, tubes were incubated at room temperature for 20 minutes. Meanwhile, cells were trypsinized, pelleted and resuspended in fresh RPMI without HERCEPTIN. 5×10⁵ cells in 2.3 ml RPMI were combined with the OptiMEM with siRNA/HiPerfect complexes and added to a well of a 6 well plate. Two days later, cells were re-transfected by the same method, and plated in 96 well plates, at 10,000 cells/well, five wells/condition. Leftover cells were plated in six well plates. The following day, zero hour counts were taken, and medium was changed in the remaining wells to RPMI containing HERCEPTIN to final concentrations of 0, 0.01, 0.03, 0.1, 0.3, and 1 ug/ml. The following day, cells plated in 6 well plates were harvested and pelleted for Western analysis to evaluate siRNA efficacy. Three days later, cells were counted, and Percent Normalized Growth was calculated.

### Example 10

Disabling MUC1* with an Anti-MUC1* Fab restores the therapeutic effect of HERCEPTIN on BTRes1 and BTRes2.

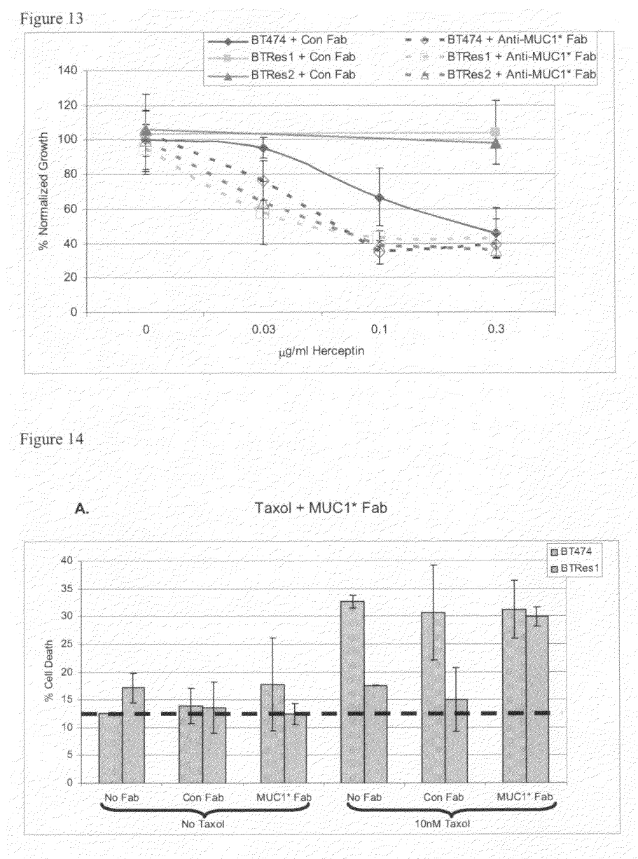

In the presence of 2.5 ug/ml of a nonspecific Fab, growth of BTRes1 and BTRes2 is unaffected by HERCEPTIN (solid lines). In the presence of an Anti-MUC1* disabling Fab, growth inhibition mediated by HERCEPTIN is restored (lower two dashed traces). For comparison, growth inhibition of BT474 cells by HERCEPTIN treatment in the presence of control Fab is identical to growth inhibition by HERCEPTIN alone (dotted line) (See FIG. 13). The experiments were performed as follows.

Growth of HERCEPTIN-Resistant BT474 Cells in the Presence of HERCEPTIN and an Anti-MUC1* Fab

BT474, BTRes1 and BTRes2 Cells were plated in 96 well plates at 10,000 cells/well, six wells/condition. The following day, zero hour counts were taken, and medium was changed in the remaining wells to RPMI containing HERCEPTIN to final concentrations of 0, 0.03, 0.1, and 0.3 ug/ml, in the presence of 2.5 ug/ml Anti-MUC1* Fab (Minerva Biotechnologies, Mahanta, et al, 2008; added to BTRes1 and BTRes2), or 2.5 ug/ml Control Fab (Jackson Immunoresearch 315-007-008; added to BT474, BTRes1, and BTRes2). One set of wells was left untreated. Three days later, cells were counted, and Percent Normalized Growth was calculated.

### Example 11

HERCEPTIN resistant cancer cells are also resistant to standard chemotherapy drugs, and this resistance is reversed by Anti-MUC1* Fab.

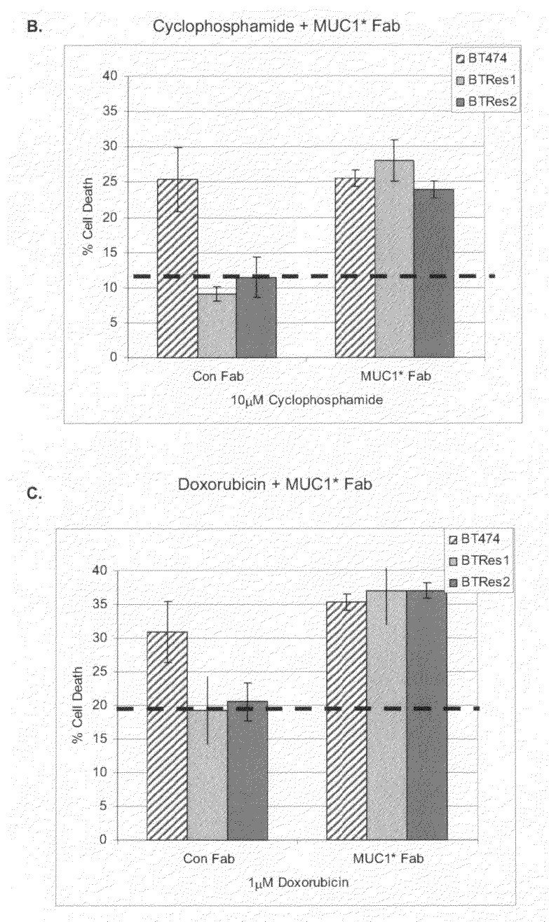

FIG. 14 shows that (A) Original BT474 cells are effectively killed by 10 nM Taxol (lined bars), as determined two days post-treatment by Trypan Blue exclusion. However, HERCEPTIN resistant cells, BTRes1 (solid bars), are essentially unaffected by Taxol when compared to untreated cells. The killing effect of Taxol is restored when the drug resistant cells, BTRes1, are treated with Taxol and 10 ug/ml of Anti-MUC1* Fab. This was also observed with 10 uM Cyclophospharnide (B) and 1 uM Doxorubicin (C) (See FIG. 14). The experiments were performed as follows.

Survival of HERCEPTIN-Resistant BT474 Cells in the Presence of Chemotherapeutic Agents Plus Anti-MUC1* Fab.

BT474, BTRes1 and BTRes2 cells were plated at 10,000 cells/well in 96 well plates, 4 wells/condition. The following day, Cyclophosphamide, Taxol, or Doxorubicin were added in varying concentrations, alone, or in the presence of 10 ug/ml Anti-MUC1* Fab or 10 ug/ml Control Fab. Cells were left untreated, or treated with either Fab alone as controls. Two days later, cells were resuspended in 50 ul trypsin, and counted in the presence of trypan blue. Percent cell death was calculated as percent trypan blue uptake.

### Example 12

Down regulation of MUC1 levels by stable expression of MUC1 shRNA sensitizes T47D cells to growth inhibition by HERCEPTIN.

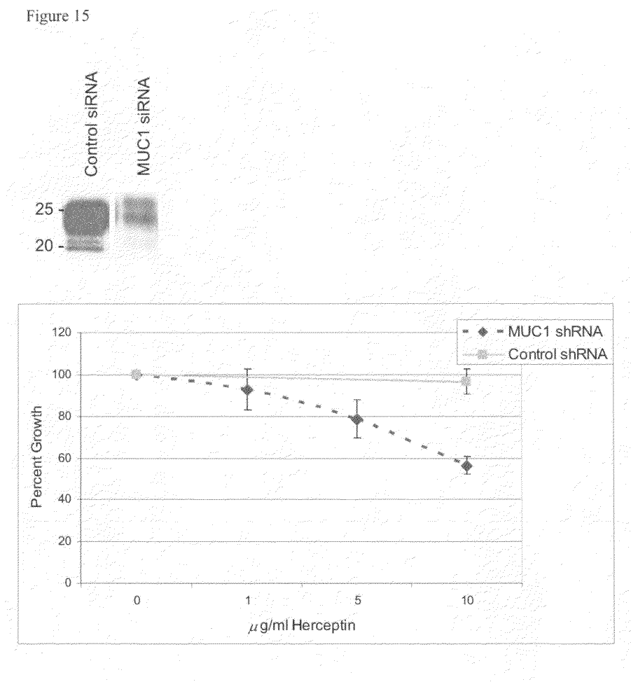

Growth of control shRNA-expressing T47D cells is unaffected by HERCEPTIN treatment (solid line), but growth of MUC1 shRNA-expressing cells is reduced in a dose-dependent manner (dotted line), see FIG. 15. The experiments were performed as follows.

shRNA-Expressing T47D Cells

T47D breast cancer cells have been reported to be intrinsically resistant to HERCEPTIN even though these cells express significant amounts of the HER2 receptor which HERCEPTIN disables. In this experiment, we show that interference RNA carried on a plasmid (shRNA) suppressed the amount of MUC1* that the cells produced and thus made the cells susceptible to treatment with HERCEPTIN. T47D cells were stably transfected with recombinant pSilencer 3.1-H1 puro plasmid (Ambion, Applied Biosystems USA) containing siRNA inserts using Lipofectin (Invitrogen). The sequence of the MUC1-specific siRNA hairpin was as follows: 5′-GCAGCCTCTCGATATAACC-ATCTCGAGG-GGTTATATCGAGAGGCTGC-3′ (SEQ ID NO:6). The hairpin sequence is in italics and the sense and the antisense 19 nucleotide siRNA sequence appears from 5′ to 3′. We used the pSilencer puro Negative Control Plasmid supplied by the manufacturer as our negative control for siRNA effect. This plasmid encodes a hairpin siRNA whose sequence is not found in the human, mouse or rat genome databases. Transfected cells were selected with media containing 0.5 μg/ml puromycin. Knock down of MUC1 protein was evaluated by Western analysis using Anti-MUC1* antibody. The T47D MUC1-specific clone is a single cell clone isolated from this pool from cells, sorted into 96-well plates by a BD Aria cell sorter (Becton Dickinson). Cells were cultured in RPMI medium as above, supplemented with 0.5 μg/ml puromycin (Calbiochem 540411).

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A colloidal nanoparticle comprising a therapeutic nucleic acid species and a targeting protein species attached via a coating on the nanoparticle that facilitates the specific attachment of both species.

2. The nanoparticle as in claim 1, wherein the nanoparticle is gold.

3. The nanoparticle as in claim 1, wherein the nanoparticle is coated with a SAM.

4. The nanoparticle as in claim 3, wherein the SAM comprises nucleic acid-thiol or nucleic acid-disulfide hybrid species and a thiol or disulfide species that facilitates the covalent attachment of proteins.

5. The nanoparticle as in claim 3, wherein the SAM comprises nucleic acid-thiol or nucleic acid-disulfide hybrid species and a thiol or disulfide species that facilitates the non-covalent attachment of proteins via affinity tags on the proteins and binding partners of the affinity tags attached to the nanoparticle.

6. The nanoparticle as in claim 1, wherein the therapeutic nucleic acid species is an interference RNA.

7. The nanoparticle as in claim 1, wherein the therapeutic nucleic acid is siRNA that has a single stranded tail hybridized to a nucleic acid incorporated into a surface coating on a nanoparticle.

8. The nanoparticle as in claim 1, wherein the therapeutic nucleic acid is siRNA that has a single stranded tail hybridized to a nucleic acid incorporated into a surface coating on a nanoparticle, the surface coating also having a functionality that facilitates the specific attachment of a targeting protein.

9. The nanoparticle as in claim 1, wherein the targeting protein is an antibody against a cell surface protein that is on the targeted cell type.

10. The nanoparticle as in claim 1, wherein the targeting protein is a peptide ligand.

11. The nanoparticle as in claim 1, wherein the coating on the nanoparticle that facilitates the specific attachment of both species, facilitates covalent coupling of an antibody to the surface.

12. The nanoparticle as in claim 1, wherein the nanoparticle surface coating comprises DNA-thiols or disulfides and an NHS-thiol or NHS-ester-thiol.

13. The nanoparticle as in claim 1, wherein the nanoparticle surface coating comprises DNA-thiols or disulfides and an NTA-Ni-thiols.

14. The nanoparticle as in claim 1, wherein the targeting antibody is HERCEPTIN and the siRNA is specific for the suppression of the MUC1 gene.

15. A method for inhibiting tumor growth comprising contacting the nanoparticle according to claim 1 with the tumor, wherein the protein target is a ligand for a receptor specifically expressed on a tumor, and wherein the therapeutic nucleic acid is interference RNA, which suppresses a tumor causing protein.

16. The method according to claim 15, wherein the tumor is breast tumor, the targeting protein is HERCEPTIN® and the therapeutic nucleic acid is interference RNA, which suppresses MUC1 expression.

17. The method according to claim 15, wherein the targeting protein is specific to MUC1* receptor.

18. The method according to claim 17, wherein the therapeutic nucleic acid is interference RNA, which suppresses MMP-14, TACE or NM23.

19. A method for modulating stem cell differentiation, comprising contacting the stem cells with the nanoparticle according to claim 1, wherein the therapeutic nucleic acid is interference RNA, which suppresses MUC1 associated protein expression.

20. A method for modulating stem cell differentiation, comprising contacting the stem cells with the nanoparticle according to claim 1, wherein the therapeutic nucleic acid is interference RNA, which suppresses gene expression of MUC1 modifying proteins.

21. The method according to claim 19, wherein the MUC1 modifying protein is MMP14, TACE, or NM23.
